(19) United States
(12) Patent Application Publication
(10) Pub. No.: US 2026/0102569 A1
(43) Pub. Date: Apr. 16, 2026

(54) Injection Device and Method of Preparing an Injection Device for Injection

(71) Applicant: Sanofi, PARIS (FR)

(72) Inventors: Steffen Raab, Frankfurt am Main (DE); Udo Stauder, Frankfurt am Main (DE); Axel Teucher, Frankfurt am Main (DE); Jean-Michel Willière, Frankfurt am Main (DE)

(21) Appl. No.: 19/116,725

(22) PCT Filed: Oct. 4, 2023
(86) PCT No.: PCT/EP2023/077368

§ 371 Date: Mar. 28, 2025

(30) Foreign Application Priority Data
Oct. 4, 2022 (EP) 22315228.1

(51) Int. Cl. A61M 5/315; A61M 5/24; A61M 5/31

(52) U.S. Cl. CPC A61M 5/31525; A61M 5/2459; A61M 5/3146

(57) ABSTRACT

An injection device for setting and injecting a dose of a medicament includes a housing; a medicament container containing the medicament and being sealed by a movable stopper as the movable stopper moves towards a proximal direction, the medicament container comprising an outlet at a distal end; and a drive mechanism arranged inside the housing and comprising a piston rod extending along a longitudinal direction and being operable to exert a distally directed pressure onto the stopper for expelling the dose of the medicament through the outlet, wherein in a pre-use configuration of the injection device, prior to expelling of a first dose of the medicament and/or prior to a first use of the injection device, the piston rod is longitudinally aligned with and abuts the stopper in a mechanically biased configuration.

### CROSS REFERENCE TO RELATED APPLICATIONS

The present application is the national stage entry of International Patent Application No. PCT/EP2023/077368, filed on Oct. 4, 2023, and claims priority to Application No. EP 22315228.1, filed on Oct. 4, 2022, the disclosures of which are incorporated herein by reference.

### TECHNICAL FIELD

The present disclosure relates to the field of injection devices, in particular to the field of pen-type injectors for setting and injecting one or multiple doses of a medicament. In another aspect the disclosure relates to a method of preparing an injection device for injecting the medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In his, e.g. in form of cartridges widely used with e.g. handheld injection devices include a tubular barrel sealed in proximal direction by a movable stopper, piston or bung. A piston rod of a drive mechanism of such injection devices is configured to advance in a distal direction thereby urging the stopper in this direction as well so as to increase a fluid pressure inside the medicament container which leads to the dispensing or injecting of the medicament via a distally located outlet, which is typically in fluid communication with an injection needle.

Due to inevitable manufacturing and/or assembly tolerances there may for instance arise axial clearance between such a piston or stopper of the medicament container and the piston rod. Typically, prior to a primary use of the device, an end-user has to conduct a so-called priming of the drive mechanism in order to ensure, that already with an initial dose setting and a subsequent dose dispensing step, an accurate amount of the medicinal product is disposed in a predefined way. for this, the user is typically instructed to the dial to set a dose of limited size into conduct an initial priming shot or an air shot by way of which a limited amount of the medicament may be expelled through the injection needle. After conducting such a priming step or procedure the device is then ready to use.

With some devices, such a priming procedure only has to be conducted before the device with the medicament container assembled therein is used for the first time. Once a priming procedure has been executed the injection device can be used repeatedly without such supplemental priming procedure is between individual dose injection procedures.

Nonetheless, some users are inclined to conduct such a priming prior to each repeated use of the injection device. Such repeated priming procedures are somewhat unnecessary and come along with a waste of medicament. At the end, and with a multiplicity of unnecessarily conducted priming procedures there may not remain a sufficient amount of medicament in the medicament container for setting and dispensing of a final dose. With such an incorrect or unintended use of the injection device there may arise a significant risk of underdosing.

It is therefore desirable to provide an improved injection device allowing for an improved and simplified use thereof especially for preparing the device prior to conduct a dose setting and injection procedure. In particular, the process of priming the injection device should be simplified for the end consumer or patients.

### SUMMARY

In one aspect, the present disclosure relates to an injection device for setting and injecting a dose of a medicament. The injection device includes a housing and a medicament container. The medicament container contains an injectable medicament. The medicament container is sealed by a movable stopper towards a proximal direction and further includes an outlet towards a distal end.

The Injection device further includes a drive mechanism arranged inside the housing and including a piston rod extending along a longitudinal direction. The piston rod and/or the drive mechanism is operable to exert a distally directed pressure onto the stopper for expelling the dose of the medicament through the outlet of the medicament container.

The injection device is transferable into a pre-use configuration. When in the pre-use configuration the piston rod is in a longitudinal and mechanically biasing abutment with the stopper. Hence, in the pre-use configuration the drive mechanism is biased or pre-tensed. Here, the piston rod is in biasing and hence pre-tension abutment with the stopper of the medicament container. In effect and when in the pre-use configuration any longitudinal tolerances and/or any longitudinal mechanical play of the drive mechanism is eliminated in such a way that an initial and distally directed displacement of the piston rod unalterably transfers into a respective distally directed movement of the stopper of the medicament container.

Typically, the injection device is in the pre-use configuration prior to expelling of a first dose of the medicament and/or prior to a first use of the injection device.

With some examples the injection device is subject to transportation and/or storage when and as long as in the pre-use configuration, or, in other words the injection device is and remains in the pre-use configuration at least during transportation and/or storage. When in the pre-use configuration the drive mechanism or the entire drivetrain provided by the drive mechanism and the longitudinal abutment with the stopper of the medicament container is longitudinally biased or longitudinally pre-tensed without having dispensed or expelled a portion of the injectable medicament through the outlet of the medicament container. Hence, in the pre-use configuration the medicament container is in a completely filled configuration and the entire medicament initially filled or disposed inside the medicament container is still located or accommodated inside the medicament container.

With some examples the medicament container includes a tubular-shaped barrel sealed by the stopper towards a proximal longitudinal end of the barrel. A distal longitudinal end of the medicament container is provided with the outlet, which may be sealed by a seal, e.g. in form of an elastomeric sealing disc. Here, the medicament container may be implemented as a cartridge, wherein the outlet is sealed by an elastomeric seal and is configured to be punctured or penetrated by a double-tipped injection needle.

Typically, and as long as the injection device is in the pre-use configuration the seal at the outlet of the medicament container is non-punctured or non-penetrated. The seal is completely intact and has not yet been penetrated by any injection needle or the like puncturing structure.

With the present injection device and with transferring the injection device into the pre-use configuration prior to use the injection device a priming step of the drive mechanism and/or of the medicament container does not or no longer have to be conducted by a user of the injection device. When the injection device is in the pre-use configuration it may be simply required by the patient or user of the injection device to attach a needle assembly to the outlet of the medicament container. An eventual raised pressure level inside the medicament container, which might be due to the longitudinal biasing abutment between the piston rod and the stopper may then relieve, thus leading to an eventual expelling of a small amount, e.g. few drops, of the liquid medicament through the outlet and hence through the injection needle of the needle assembly. Nonetheless and when in the pre-use configuration a separate user-induced execution of a priming procedure or of an air shot is no longer required.

When the injection device is handed out to patients or users in the pre-use configuration and when the user or patient has duly attached an injection needle to the outlet of the medicament container an increased fluid pressure inside the medicament container may induce an expelling of a small amount, e.g. a few drops of the liquid medicament.

In any case and due to the longitudinal and mechanically biasing abutment between the piston rod and the stopper, the drive mechanism or the injection device is void of any longitudinal tolerances in the drive train of the drive mechanism so that with a subsequent and first or initial setting and injecting procedure to be conducted with the drive mechanism a set dose can be completely dispensed or injected.

Thus, with the presently proposed injection device a priming, mechanical biasing or pre-tensioning of the drive mechanism thereby bringing the piston rod in longitudinal and mechanically biasing abutment with the stopper of the cartridge, can be conducted at the end of manufacturing or at the end of assembly of the injection device e.g. by a pharmaceutical manufacturer. The injection device may remain in the pre-use configuration during transportation and storage to patients or customers. Accordingly, and when a patient or user intends to use the injection device for the first time it is no longer required to instruct the patient or user to conduct or to execute a separate priming procedure. For the end user or patient it will be only required to correctly assemble a needle assembly to the housing and/or to the outlet of the medicament container so as to transfer the injection device from the pre-use configuration into a ready to use configuration.

According to a further example, and when in the pre-use configuration, the piston rod exerts a distally directed pre-use pressure onto the stopper. The pre-use pressure is larger than or equal to a driving pressure, which is required to move the stopper in distal direction for injecting of the dose of the medicament.

Upon applying of a pre-use pressure onto the stopper the stopper may not move in distal direction despite the fact that the pre-use pressure is larger than the driving pressure, which is required to move the stopper in distal direction. Here, and as long as the injection device is in the pre-use configuration the outlet of the medicament container may be and remain sealed.

Due to a substantial incompressibility of the e.g. liquid medicament contained in the medicament container application of a comparatively large pre-use pressure onto the stopper in distal direction may not have a substantial effect besides a longitudinal compression of the stopper, which is governed by the elastic material of the stopper, this leading to the generation of an increased pressure inside the medicament container.

It may be only upon attaching of an injection needle, e.g. a double-tipped injection needle, to the outlet of the medicament container that the pre-use pressure applied by the piston rod onto the stopper of the medicament container leads to a dispensing of a comparatively small amount of the medicament through the outlet.

According to a further example the distal end of the medicament container is in longitudinal abutment with a stop face of the housing. Typically, the stop face of the housing is provided at or near a distal end of the housing. The housing of the injection device may include a single or multiple housing components. With multiple housing components the housing may include a main housing component, also denoted as a body of the injection device forming a proximal housing component. A further housing component of the injection device may provide a cartridge holder or cartridge retainer. It may form or constitute a distal housing component of the injection device.

The cartridge holder or cartridge retainer portion of the housing of the injection device may include a mechanical connector at a distal end complementary shaped to a counter connector of a standardized needle assembly. The cartridge holder is sized and configured to accommodate the medicament container, e.g. in form of the cartridge. The body of the housing is configured and sized to accommodate the drive mechanism. The proximal end of the cartridge holder may be detachably or non-detachably connectable or fastenable to a distal end of the body of the housing. Typically, and in a final stage of assembling the injection device the cartridge or medicament container is inserted in distal direction through the proximal end of the cartridge holder and the cartridge holder with the cartridge or medicament container assembled therein is then connected and fixed to the distal end of the body. Due to inevitable geometric metric tolerances or assembly tolerances of numerous components of the injection device there may arise a a rather loosely fitting between the piston rod and the stopper and/or a longitudinal gap between a proximal face of the stopper of the medicament container or cartridge and a distal end of the piston rod. Then and after mechanically fixing the cartridge holder to the body of the injection device the drive mechanism may be used to advance the piston rod in distal direction until it reaches a mechanically biasing abutment with the stopper, thereby transferring the injection device into the pre-use configuration.

With some examples a distally directed stop face of the medicament container, e.g. a distally facing shoulder portion of the medicament container may be in longitudinal abutment with a complementary shaped proximally facing stop face of the housing or of the cartridge holder. In this way, the medicament container or cartridge is confined in distal direction relative to the housing or cartridge holder. Due to the longitudinal biasing abutment between the stopper of the cartridge and the piston rod the medicament container is also longitudinally confined with respect to the proximal direction relative to the housing of the injection device.

According to a further example and when in the pre-use configuration the outlet of the medicament container is sealed and is impenetrable for the medicament. In the pre-use configuration and as long as the injection device is in the pre-use configuration the outlet remains sealed and also remains substantially impenetrable for the medicament. Here, exertion of a comparatively high pressure or trust in distal direction onto the stopper of the medicament container does not lead to the expelling of the medicament because the outlet of the medicament container is and remains sealed.

The pre-use configuration may be abrogated or the injection device may be transferred from the pre-use configuration into a ready to use configuration simply by attaching a needle assembly to the distally located outlet, thereby penetrating the seal of the medicament container by e.g. an injection needle.

According to a further example and when the injection device is in the pre-use configuration the outlet of the medicament container is and remains non-punctured. Here, the outlet may be covered or closed by a pierceable seal, such as an elastomeric sealing disc also denoted as or including a rubber septum. As long as the injection device is in the pre-use configuration the seal is undamaged and is non-punctured.

Transferring of the injection device from the pre-use configuration into the ready to use configuration may be accompanied by piercing the seal of the outlet e.g. by a double-tipped injection needle.

According to a further example the medicament container includes a barrel of e.g. tubular shape. The outlet of the medicament container is sealed by a needle-penetrable elastomeric seal. Here, the outlet of the medicament container is provided by an outlet of the barrel. Hence, the needle penetrable elastomeric seal seals and closes the outlet of the barrel of the medicament container. The barrel may be of substantially tubular shape. It may include a radially narrowing or radially stepped down neck portion towards its distal end and hence towards the outlet. The outlet e.g. provided at the distal end of the barrel may include a beaded cap. Here, the elastomeric seal, e.g. in form of a pierceable sealing disc is kept and fixed to a head portion provided at the distal end of the barrel of the medicament container.

According to a further example the medicament container includes a seal closing or sealing the outlet of the medicament container. The seal is fixed to the distal end of the medicament container by a fixing cap. The fixing cap includes an outlet aperture to provide access to a pierceable portion of the seal. Typically, the pierceable portion of the seal is a radial central portion of the seal. The fixing cap may be a crimped cap or a beaded cap covering or enclosing a distal free end of the barrel of the medicament container. The fixing cap may be made of a pliable material, such as a pliable metal sheet material, e.g. aluminum.

The fixing cap provides a liquid-proof and durable fixing of the penetrable seal to the outlet of the medicament container. In the same way and since the pierceable portion of the seal is and remains uncovered and is hence accessible through the outlet aperture of the fixing cap there is provided a direct access, e.g. for a double-tipped injection needle to penetrate the seal and to thereby obtain a fluid transferring access to the interior of the medicament container. The seal as provided and/or fixed at the outlet of the medicament container may be elastically deformable.

According to a further example and when in the pre-use configuration the pierceable portion of the seal includes or forms a bulged seal portion at least partially protruding distally through the outlet aperture of the fixing cap. This bulged seal portion may arise due to the application of the pre-use pressure onto the stopper. Here, the stopper may be subject to longitudinal compression. Since the piston rod applies the pre-use pressure onto the stopper the stopper itself may be also subject to a distally directed movement relative to the sidewall of the medicament container, e.g. relative to the barrel of the container thereby increasing the fluid pressure and thereby inducing an outwardly bulged deformation of the seal by way of which the pierceable portion of the seal starts to protrude distally through the outlet aperture.

Generation of such an outwardly extending or outwardly protruding pierceable portion of the seal provides a visible or tactile feedback to a user that the injection device is in the pre-use configuration. With some examples the distal end of the housing of the injection device, e.g. a distal end of the cartridge holder includes an aperture to receive the injection needle there through. The outwardly bulged seal portion may be at least visible through this aperture if not palpable in the aperture of the cartridge holder or housing.

By way of the at least partially and distally protruding bulged seal portion of the medicament container the patient or user of the injection device may check visually or haptically if the injection device is in the intended pre-use configuration before a respective injection needle is attached to the distal end of the injection device.

According to another example and when in the pre-use configuration a hydrostatic pressure inside the medicament container is larger than atmospheric pressure. Hence, when in the pre-use configuration the interior of the medicament container is subject to an increased pressure compared to atmospheric pressure.

This increased pressure level may give rise to the formation or generation of the bulged seal portion of the pierceable portion of the seal of the medicament container or cartridge.

According to a further example of the injection device and when in the pre-use configuration the hydrostatic pressure inside the medicament container exceeds the atmospheric pressure by a factor p, wherein p>1.1, p>1.2, p>1.3, p>1.4, p>1.5, p>1.6, p>1.7, p>1.8, p>1.9, p>2.0, p>2,1, p>2.2, or p>2.3. With such a pressure increase a well-defined level pre-tension can be obtained which pressure increase is sufficient to eliminate longitudinal tolerances and/or mechanical play in the injection device and the drive mechanism. On the other hand the pressure increase is well below an upper threshold level at which the mechanical structure of the injection device, of the drive mechanism or of the medicament container could become subject to mechanical damage.

According to another example and when in the pre-use configuration the piston rod of the drive mechanism is in longitudinal abutment with the movable stopper and of the medicament container and applies a longitudinally directed pressure onto the stopper in a range between 10 kPa and 130 kPa. Hence, there may be a well-defined longitudinal or axial abutment between the piston rod and the stopper with an abutment pressure in a range between 10 kPa and 130 kPa.

With some examples, the abutment pressure is in a range between 20 kPa and 100 kPa. With some examples the abutment pressure is in a range between 40 kPa and 80 kPa. With some examples the abutment pressure is in a range between 50 kPa and 70 kPa.

With further examples the longitudinal abutment pressure between the piston rod and the stopper is larger than 10 kPa, larger than 20 kPa, larger than 30 kPa, larger than 40 kPa, larger than 50 kPa, larger than 60 kPa, larger than 70 kPa, larger than 80 kPa, larger than 90 kPa, larger than 100 kPa, larger than 110 kPa, or larger than 120 kPa.

The longitudinal abutment pressure between the piston and the piston rod may be adjusted in accordance to the type of the medicament, in particular in accordance to the viscosity of the medicament and/or with regard to the geometric dimensions or material properties of the medicament container and the stopper.

According to another example of the injection device and when in the pre-use configuration the piston rod is in a priming position relative to the housing, which priming position is distally offset compared to an initial position of the piston rod upon assembly or upon final assembly of the injection device. The piston rod may be easily transferred from the initial piston rod position into or towards the pre-use position simply by conducting a kind of a priming action of the drive mechanism without needle attachment.

Here, a dose of a predefined size, e.g. a dose of 1-5 units may be set by the drive mechanism and the respective dose may be only virtually injected by conducting a respective injection or dispensing procedure thus leading to a respective distally directed displacement of the piston rod in accordance to the predefined size of a dose previously set. In this way, comparatively small the dose sizes, such as 1, 2 or up to 5 international units of the medicament may be set and the drive mechanism may be subsequently triggered to inject such a dose thereby moving the piston rod from the initial position towards and into the priming or pre-use position, in which the piston rod gets into the above-mentioned longitudinal and mechanically biasing abutment with the stopper. During this virtual injection procedure the outlet of the medicament container is and remains sealed and expelling of the liquid medicament is effectively prevented.

In another aspect the present disclosure relates to an injection system. The injection system includes a packaging providing a receptacle for an injection device. The injection system further includes an injection device as described above, which is arranged inside the receptacle in its pre-use configuration. The injection system, i.e. the packaging with the injection device disposed therein may be intended for transportation and/or storage of the injection device.

By keeping the injection device in its pre-use configuration inside the packaging the user does not have to conduct a separate priming procedure with the injection device prior to a first use. Rather, and for transferring the injection device from a pre-use configuration into a ready to use configuration the user simply has to attach or to assemble a suitable needle assembly to the injection device. During or after attaching the needle assembly to the injection device it may be provided that a proximal end of a double-tipped injection needle extends through an aperture at the distal end of the injection device and penetrates the seal of the medicament container.

Due to the increased pre-use pressure inside the medicament container a comparatively small amount of medicament may be initially expelled during the step of needle attachment. Since the piston rod of the drive mechanism is and remains in a longitudinal abutment with the stopper the injection device is in a ready to use configuration right after attachment of the needle assembly.

The injection system contains the injection device as described above. Insofar, all features, effects and benefits as described above in connection with the injection device equally apply for the injection system.

The packaging may include one of a blister packaging, a foil-type packaging or a cardboard packaging. The packaging may contain a single or a plurality of injection devices. With some examples the packaging is non-transparent. With other examples packaging or a portion thereof is translucent. With a packaging being opaque especially for electromagnetic radiation in the visible spectral range and/or in any of the infrared or UV spectral range the medicament contained in the medicament container can be effectively protected against such radiation thereby allowing enabling to extend the storage or shelf life of the medicament located inside the medicament container.

With further examples the packaging is liquid-proof and/or gas-proof. It may be thus impervious to liquid substances, gases or gaseous substances. In this way, the injection device can be effectively protected against further environmental influences, such as humidity and/or dust.

In another aspect the present disclosure also relates to a method of preparing an injection device for injecting a medicament. The method includes the steps of providing an injection device, typically to provide an injection device as described above. Subsequently, the method includes the step of transferring the injection device into the above-mentioned pre-use configuration by moving the piston rod in distal direction against the stopper of the medicament container.

Typically, the advancing motion of the piston rod is such that the piston rod exerts a well-defined pressure to the stopper of the piston rod. With some examples the advancing of the piston rod in distal direction continues or is continued until a predefined pressure level been reached between the piston rod and the stopper. With some examples the advancing motion of the piston rod is pressure-controlled. With other examples the piston rod is advanced in distal direction by a predefined distance, wherein the predefined distance is larger than the longitudinal tolerance margins between the distal end of the piston rod and the proximal end of the stopper upon final assembly of the injection device.

In this way and when advancing the piston rod for a predefined distance along the distal direction after final assembly of the injection device it can be somehow guaranteed that the piston rod is not only in longitudinal abutment with the stopper but that the piston rod also applies a well-defined pressure to the stopper above a predefined threshold.

The pressure threshold may be in a range, which is above a driving pressure normally required to move the stopper in distal direction for injecting of the dose of the medicament when an injection needle is attached to the injection device and when the medicament can be expelled through the outlet of the medicament container through the injection needle.

As described above and in the pre-use configuration the medicament container is and remains sealed at its outlet so as to prevent an uncontrolled dispensing or expelling of the medicament from the medicament container.

The method of preparing the injection device is particularly intended to be executed or to be conducted by an injection device as described above. Insofar, all effect, features and benefits as described above in connection with the injection device equally apply for the method of preparing an injection device.

Transferring the injection device into the pre-use configuration may be conducted by a pharmaceutical manufacturer. Here, transferring of the injection device may be conducted as a final step after a final assembly of the injection device on-site by the pharmaceutical manufacturer. The injection device in its pre-use configuration may be stored and shipped or transported to consumers and/or patients. The consumers or patients then do no longer have to conduct a separate priming procedure. Rather, it will be only necessary to attach a needle assembly to the injection device in its pre-use configuration thereby automatically transferring the injection device into the ready to use configuration.

Transferring the injection device into the pre-use configuration prior to packaging and shipment of the injection device to consumers or patients has a further benefit to firmly fix the medicament container inside the housing of the injection device. In this way, the medicament container can be kept slack-free inside the housing of the injection device during transportation and/or shipment. Moreover and with the mechanically biased abutment between the piston rod and the stopper the entire drive mechanism can be kept in a somewhat pre-tensed or pre-biased state thus inhibiting any motion of loosely fitted components of the drive mechanism, which otherwise may be subject to vibration or comparable tiny movements during transportation or shipment.

According to a further example the method of preparing the injection device further includes the step of attaching a needle assembly to the injection device after transferring the injection device into the pre-use configuration. Typically, the needle assembly includes a double-tipped injection needle with a proximal end and a distal end. The injection needle is tipped at both of its distal end and its proximal end. Upon attaching the injection needle to the injection device the proximal end of the injection needle penetrates a seal provided at the outlet of the medicament container.

According to a further example the proximal end of the injection needle penetrates the seal of the medicament container upon attaching the needle assembly to the injection device.

Typically, the needle assembly includes a needle hub or needle holder, which may be of somewhat tubular shape. The needle hub typically includes a counter connector complementary shaped to a connector at the distal end of the injection device. The connector and the counter connector may be implemented as an outer thread and a mating inner thread, respectively. With other examples the connector and the counter connector may form a snap fit connection or a bayonet connection.

The injection needle is typically removably fastened to the needle holder. By attaching the needle, in particular by attaching the needle hub to the distal end of the injection device a fluid transferring connection is established between the injection needle and an interior of the medicament container. This way and upon attaching the injection needle to the injection device the injection device may transfer into a ready to use configuration. Due to the mechanically biasing abutment between the piston rod and the stopper there may be an increased hydrostatic pressure inside the medicament container before attaching the needle assembly. Upon completion of the attachment of the needle assembly to the injection device there may arise a kind of a pressure relief thus leading to the expelling of a limited amount of the medicament through the injection needle.

With a further example of the method and in the course of attaching the needle assembly to the injection device a portion of the medicament is expelled through the injection needle due to a distally directed pre-use pressure exerted by the piston rod onto the stopper in the pre-use configuration.

Application of the pre-use pressure onto the stopper may lead to an increase of the hydrostatic pressure inside the medicament container as long as the injection needle is not attached to the medicament container or to the injection device. Application of the distally directed pre-use pressure onto the stopper may also lead to a longitudinal compression of the stopper due to the elastic properties of the stopper. Since the liquid medicament contained inside the medicament container is substantially incompressible the increased pressure applied to the stopper may lead to a radial widening of the stopper thereby increasing a sealing function of sealing capability of the stopper. Longitudinal compression of the stopper may therefore lead to a radial widening of the stopper by way of which the stopper applies an increased radially outwardly directed pressure to the sidewall of the tubular shaped barrel of the medicament container.

In this way and as long as the injection device is and remains in the pre-use configuration there may be provided an increased sealing capability for the stopper.

According to another example the needle assembly includes at least one of an outer needle cap and an inner needle cap covering the distal end of the injection needle. The outer needle cap or the inner needle cap are further configured to receive or to trap the portion of the medicament, which is expelled through the distal end of the injection needle in the course of attaching the needle assembly to the injection device when the injection device is in its pre-use configuration. A portion of the medicament, e.g. being automatically expelled due to the attachment of the needle assembly to the injection device may be then trapped inside the outer needle cap or inner needle cap and the patient may not even take notice of the expelling of the medicament.

Moreover, a pressure relief leading to the expelling of a small amount of the medicament through the injection needle in the course of assembling the injection needle may take place during the needle attachment and before completion of the attachment of the needle assembly to the injection device. Hence, and when arriving at a final mutual assembly configuration of needle assembly and injection device the process of expelling a small amount of medicament from the medicament container may have already terminated.

According to a further example the injection device is transferable or is transferred from the pre-use configuration into the ready to use configuration by attaching the needle assembly to the injection device for transferring the injection device into the ready to use configuration it is no longer required that the user conducts a separate priming procedure, e.g. by dialing or setting a predefined size of a dose and by subsequently injecting the those of the medicament. It is simply required to attach the needle assembly to the injection device and to eventually trap a particular amount of the medicament expelled through the injection needle.

When the user then intends to use the injection device and removes the inner needle cap and/or the outer needle cap there will no longer appear any drops at the distal end of the injection needle. In this way, the patient or user of the injection device may not even become aware that a priming procedure or an air shot has been somewhat automatically conducted in order to compensate or to eliminate any tolerances or mechanical play in the drive train of the drive mechanism of the injection device.

According to a further example the injection device is arranged inside a packaging for transportation and/or storage after being transferred into the pre-use configuration. Hence, the method of preparing the injection device may be conducted on site of a pharmaceutical manufacturer. After a final assembly of the injection device it may be transferred into its pre-use configuration. Thereafter, the injection device may be packed or wrapped inside the packaging thereby forming an injection system.

The method of preparing the injection device may be continued by the end user or patient. Here, the injection device may be unwrapped or released from the packaging and a needle assembly will be duly attached to the injection device. As a further optional method step at least one of an outer needle cap and an inner needle cap may be detached from the needle assembly, after the needle assembly has been attached and fixed to the injection device. The injection device is then in a ready to use configuration in which a user may subsequently set and inject a single or multiple doses of the medicament.

Since with the present injection device it is not intended to conduct a user-controlled priming procedure and since the user is not at all instructed to set a dose and to subsequently trigger an injection procedure for the purpose of priming such superfluous and potentiality medicament-wasting dispensing procedures between repeated dose injection procedures, potential misuse of the injection device can be effectively avoided.

Generally, the scope of the present disclosure is defined by the content of the claims. The disclosure is not limited to specific embodiments or examples but includes any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about 4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(107 -carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE FIGURES

In the following, examples of injection devices and a method of preparing the same for injection will be described in greater detail by making reference to the drawings, in which:

FIG. 1 schematically illustrates a longitudinal cross-section through an example of an injection device,

FIG. 2 shows a distal end of a medicament container when the injection device is in the pre-use configuration,

FIG. 3 shows the medicament container according to FIG. 2 after attachment of a needle assembly to the injection device,

FIG. 4 is illustrative of the injection device and the needle assembly,

FIG. 5 is a further illustration of the injection device with a cartridge holder detached from a body of the injection device,

FIG. 6 is a flowchart of a method of preparing an injection device for injecting a medicament, and

FIG. 7 illustrates an injection device arranged in a packaging.

### DETAILED DESCRIPTION

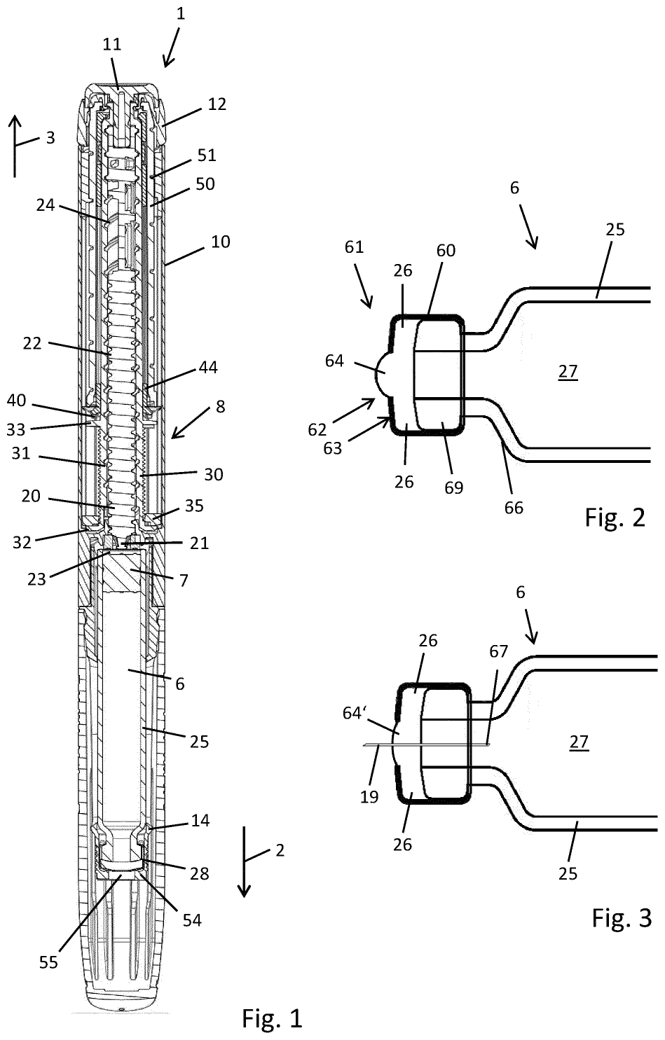

The injection device 1 as shown in FIGS. 1, 4 and 5 is a pre-filled disposable injection device that includes a housing 10 to which a needle assembly 15 can be affixed. The needle assembly 15 includes a double-tipped injection needle 19. The needle 19 protected by an inner needle cap 16 and either an outer needle cap 17 or a protective cap 18 that is configured to enclose and to protect a distal section of the housing 10 of the injection device 1. The housing 10 may include and form a main housing part configured to accommodate a drive mechanism 8. The injection device 1 may further include a distal housing component denoted as cartridge holder 14. The cartridge holder 14 may be permanently or releasably connected to the main housing 10. The cartridge holder 14 is typically configured to accommodate a medicament container 6, e.g. implemented as a so-called cartridge that is filled with a liquid medicament.

The cartridge or medicament container 6 includes a cylindrically-shaped or tubular-shaped barrel 25 sealed in proximal direction 3 by a stopper 7 located inside the barrel 25. The stopper 7 is displaceable relative to the barrel 25 of the medicament container 6 in a distal direction 2 by a piston rod 20. A distal end of the medicament container 6 is sealed by a pierceable seal 26 configured as a septum and being pierceable by a proximally directed tipped end 67 of the injection needle 19.

The needle assembly 15 includes a needle hub 70 including a counter connector complementary shaped to the connector 28 provided at the distal end of the housing 10 or the cartridge holder 14. With the presently illustrated example the connector 28 includes a threaded socket with an outer thread complementary shaped to an inner thread provided at or in the sidewall of the needle hub 70. In this way, the needle assembly 15 can be attached and detached to the distal end of the injection device 1 by screwing or unscrewing.

By attaching the needle assembly 15 to the distal end of the cartridge holder 14 the seal 26 of the medicament container 6 is penetrated by the injection needle 19 thereby establishing a fluid transferring access to the interior of the medicament container 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 ug of pure crystalline insulin ( 1/22 mg).

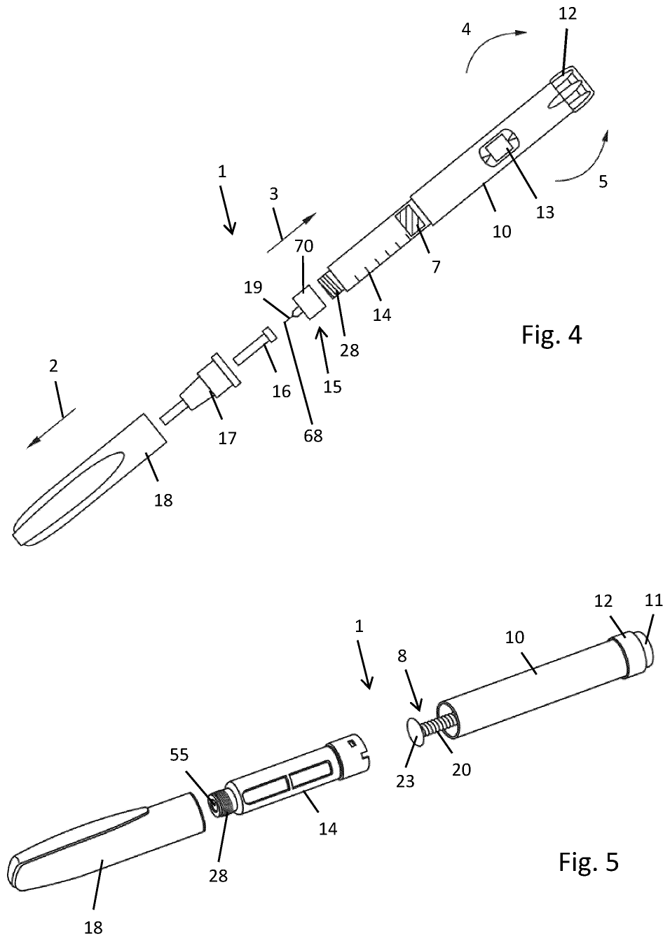

As shown further in FIGS. 1 and 4, the housing 10 includes a dosage window 13 that may be in the form of an aperture in the housing 10. The dosage window 13 permits a user to view a limited portion of a number sleeve 50 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The number sleeve 50 mechanically interacts with a piston in the medicament container 6. When the needle 19 is stuck into a skin portion of a patient, and when the trigger 11 or injection button is pushed, the dose displayed in display window 13 will be ejected from injection device 1. When the needle 19 of the injection device 1 remains for a certain time in the skin portion after the trigger 11 is pushed, a high percentage of the dose is actually injected into the patient's body. Ejection of a dose of the medicament 27 may also cause a mechanical click sound, which is however different from the sounds produced when using the dose dial 12.

In this embodiment, during delivery of the dose of the medicament, the dose dial 12 is turned to its initial position in an axial movement, that is to say without rotation, while the number sleeve 50 is rotated to return to its initial position, e.g. to display a dose of zero units.

The injection device 1 may be used for several injection processes until either the medicament container 6 is empty or the expiration date of the medicament in the injection device 1 (e.g. 28 days after the first use) is reached.

The expelling or drive mechanism 8 as illustrated in more detail in FIG. 1 includes numerous mechanically interacting components. A flange like support of the housing 10 includes a threaded axial through opening threadedly engaged with a first thread or distal thread 22 of the piston rod 20. The distal end of the piston rod 20 includes a bearing 21 on which a pressure foot 23 is free to rotate with the longitudinal axis of the piston rod 20 as an axis of rotation. The pressure foot 23 is configured to axially abut against a proximally facing thrust receiving face of the stopper 7 of the medicament container 6. During a dispensing action the piston rod 20 rotates relative to the housing 10 thereby experiencing a distally directed advancing motion relative to the housing 10 and hence relative to the barrel 25 of the medicament container 6. As a consequence, the stopper 7 of the medicament container 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 includes an inner thread threadedly engaged with the proximal thread 24 of the piston rod 20. Moreover, the drive sleeve 30 includes an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further includes a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 44. At a first end the clutch 44 is provided with a series of circumferentially directed saw teeth. Towards a second opposite end of the clutch 44 there is located a radially inwardly directed flange.

Furthermore, there is provided a dose dial sleeve also denoted as number sleeve 50. The number sleeve 50 is provided outside of the spring 40 and the clutch 44 and is located radially inward of the housing 10. A helical groove 51 is provided about an outer surface of the number sleeve 50. The housing 10 is provided with the dosage window 13 through which a part of the outer surface of the number 50 can be seen. The housing 10 is further provided with a helical rib at an inside sidewall portion, which helical rib is to be seated in the helical groove 51 of the number sleeve 50. There are provided first and second stops on the housing 10 to limit a dose setting procedure during which the number sleeve 50 is rotated in a helical motion relative to the housing 10.

The dose dial 12 in form of a dose dial grip is disposed about an outer surface of the proximal end of the number sleeve 50. An outer diameter of the dose dial 12 typically corresponds to and matches with the outer diameter of the housing 10. The dose dial 12 is secured to the number 50 to prevent relative movement therebetween. The dose dial 12 is provided with a central opening.

The trigger 11, also denoted as dose button is substantially T-shaped. It is provided at a proximal end of the injection device 10. A stem of the trigger 11 extends through the opening in the dose dial 12, through an inner diameter of extensions of the drive sleeve. The trigger 11 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 44 engaged, the drive sleeve 30, the spring or clicker 40, the clutch 44 and the number sleeve 50 rotate with the dose dial 12. Audible and tactile feedback of the dose being dialed is provided by the spring 40 and by the clutch 44. Torque is transmitted through saw teeth between the spring 40 and the clutch 44. The helical groove 51 on the number sleeve 50 and a helical groove in the drive sleeve 30 have the same lead. This allows the number sleeve 50 to extend from the housing 10 and the drive sleeve 30 to climb the piston rod 20 at the same rate. At a limit of travel a radial stop on the number sleeve 50 engages either with a first stop or a second stop provided on the housing 10 to prevent further movement in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector, allows the dosage to be dialed down without dispense of the medicament from the medicament container 6. For this the dose dial 12 is simply counter-rotated. This causes the system to act in reverse. A flexible arm of the spring or clicker 40 then acts as a ratchet preventing the spring 40 from rotating. The torque transmitted through the clutch 44 causes the saw teeth to ride over one another to create the clicks corresponding to dialed dose reduction. Typically, the saw teeth are so disposed that a circumferential extent of each saw tooth corresponds to a unit dose.

When the desired dose has been dialed the user may simply dispense the set dose by depressing the trigger 11. This displaces the clutch 44 axially with respect to the number sleeve 50 causing dog teeth thereof to disengage. However, the clutch 44 remains keyed in rotation to the drive sleeve 30. The number sleeve 50 and the dose dial 12 are now free to rotate in accordance with the helical groove 51.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 44 back along the drive sleeve 30 to restore the connection between the clutch 44 and the number sleeve 50 when the distally directed dispensing pressure is removed from the trigger 11.

The longitudinal axial movement of the drive sleeve 30 causes the piston rod 20 to rotate through the through opening of the support of the housing 10, thereby to advance the stopper 7 in the cartridge 6. Once the dialed dose has been dispensed, the number sleeve 50 is prevented from further rotation by contact of at least one stop extending from the dose dial 12 with at least one corresponding stop of the housing 10. A zero-dose position may be determined by the abutment of one of axially extending edges or stops of the number sleeve 50 with at least one or several corresponding stops of the housing 10.

During dose setting and when the drive mechanism 8 or the dose setting mechanism 9 is in the dose setting mode the drive sleeve 30 rotates in unison with the dose dial 12 and with the number sleeve 50. The drive sleeve 30 is threadedly engaged with the piston rod 20, which during dose setting is stationary with regard to the housing 10. Accordingly, the drive sleeve 30 is subject to a screwing or helical motion during dose setting. The drive sleeve 30 starts to travel in proximal direction as the dose dial is rotated in a dose incrementing direction 4, e.g. in a clockwise direction. For adjusting of or correcting a size of a dose the dose dial 12 is rotatable in an opposite direction, hence in a dose decrementing direction 5, e.g. counterclockwise.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

As it is further apparent from FIG. 1 the distal end of the cartridge holder 14 includes a proximally directed stop face 54, e.g. on the inside of an end face of the cartridge holder 14. The end face of the cartridge holder 14 may be provided with a central aperture 55. This way and when attaching a needle assembly 15 with a double-tipped injection needle 19 the proximal end 67 of the injection needle 19 may reach through the aperture 55 and may penetrate the seal 26 as provided at the outlet 61 of the medicament container 6.

With the presently illustrated example the medicament container 6 is implemented as a cartridge. The medicament container 6 includes a tubular-shaped barrel 25. Towards the distal end 63 the barrel 25 includes a radially narrowing shoulder portion 66 extending into a head portion 69 provided at the free and distal end 63 of the barrel 25. The outlet 61 of the medicament container 6 is covered and/or sealed by the pierceable seal 26, which may be fixed to the head portion 69 by a fixing cap 60. The fixing cap 60 may include or form a beaded cap providing a form-fitting of the fixing cap 60 and the seal 26 to the distal end 63 of the barrel 25. The fixing cap 60 includes a central outlet aperture 62, through which the proximal end 67 of the injection needle 15 can be guided so as to pierce or penetrate the seal 26 as illustrated in FIG. 3.

The injection device 1 as illustrated in FIG. 1 may be in a pre-use configuration as defined above. In the pre-use configuration the piston rod 20 and/or pressure foot 23 is in longitudinal and mechanically biasing abutment with the stopper 7 of the medicament container 6. The medicament container 6 is retained inside the cartridge holder 14 by a longitudinal abutment of the distal end 63 of the medicament container 6 with the proximal stop face 54 of the cartridge holder 14. In addition or alternatively, the shoulder portion 66 of the medicament container 6 is in longitudinal distal abutment with a complementary shaped proximal stop face as provided on the inside of the cartridge holder 14.

Towards the proximal direction 3 the medicament container 6 is confined and/or fixed inside the housing 10 or cartridge holder 14 due to the longitudinal abutment between the pressure foot 23 and the proximal end of the stopper 7.

Upon reaching a final assembly configuration of the injection device 1, i.e. when fixing or attaching the cartridge holder 14 with the medicament container 6 located therein to the body 10 of the injection device 1, as schematically illustrated in FIG. 5, a mutual abutment configuration between the stopper 7 and the piston rod 20 may be already obtained. However, upon reaching a final assembly configuration and during assembly of the injection device it is generally avoided to apply a substantial and distally directed pressure to the stopper 7 of the medicament container 6.

Now and with the present injection device 1 there is provided a dedicated step of transferring the injection device 1 into the pre-use configuration, namely by moving the piston rod 20 in distal direction 2 against the stopper 7 until a well-defined mechanically biasing abutment is obtained with the stopper 7.

When arriving in the pre-use configuration the piston rod 20 and/or if pressure foot 23 exerts a distally directed pre-use pressure onto the stopper 7, which is counteracted by the abutment of the distal end 63 or the shoulder portion 66 with a complementary shaped proximally facing stop face 54 of the cartridge holder 14. The pre-use pressure applied onto the stopper 7 is typically larger than or equal to a driving pressure normally required to move the stopper in distal direction for injecting of the dose of the medicament when the injection needle 19 has unhindered access to the interior of the medicament container 6. In this way and when reaching the pre-use configuration the drive train or drive mechanism 8 and the medicament container 6 are in a pre-tensed or biased state.

In the pre-use configuration the distal end 63 and hence the outlet 61 of the medicament container 6 is and remains unconnected to the needle assembly 15. As a consequence of the increased pressure level inside the medicament container the elastomeric seal 26, in particular a central portion of the seal 26 located or overlapping with the outlet aperture 62 of the fixing cap 60 may be subject to a distally protruding outwardly bulging or deformation thus leading to the formation of a respective outwardly and distally extending bulged portion 64 of the seal 26. Now and for transferring the injection device 1 from the pre-use configuration into a ready to use configuration it is intended to attach the needle assembly 15 to the distal end of the injection device 1.

During and upon attaching of the needle assembly 15 to the cartridge holder 14 the proximal end 67 of the injection needle 19 will penetrate the seal 26 as indicated in FIG. 3. As a consequence and due to the increased the fluid pressure inside the medicament container there may be a pressure relief in the medicament container 6 as soon as the proximal end 67 has traversed the seal 26. The pressure relief comes along with expelling a particular amount of the medicament through the injection needle 19.

Consequently, there may be expelled a few drops from the distal end 68 of the injection needle 19 during the process of needle assembly. As a consequence and due to the pressure relief the bulged portion 64 of the seal 26 may relax into an initial unbiased state 64′ as illustrated in FIG. 3. The elastic return motion of the seal 26 from the outwardly stretched bulged portion 64 towards the unbiased or relaxed state 64′ comes along with a slight reduction of the available storage space for the liquid medicament inside the medicament container 6. This storage space reduction may also contribute to the expelling of a limited amount of the medicament through the injection needle 19 upon needle attachment.

The amount of medicament that may be expelled due to a pressure relief during attachment of the injection needle to the injection device may be trapped in the inner needle cap 16 and/or in the outer needle cap 17 that are attached to the needle assembly 15 when attaching and fixing the needly assembly to the injection device 1. In this way and due to the needle cap 16, 17 the end user or patient may be unaware of the pressure relief action while attaching the needle assembly. The amount of medicament expelled during needle attachment may be rather elegantly trapped in the inner and/or outer needle cap 16, 17. In a final step of preparing the injection device for conducting an injection procedure the needle caps 16, 17 will have to be removed.

Transferring of the injection device from the pre-use configuration into a ready to use configuration already starts when the injection needle has penetrated the seal 26 during the assembly process of the injection needle 15. In FIG. 3 the final attachment position of the needle assembly 15 to the injection device 1 and/or to the medicament container 6 is schematically illustrated. As indicated in FIG. 3 and when reaching the final assembly position the proximal end 67 of the injection needle 19 is located at a well-defined longitudinal distance from an inside surface of the seal 26.

During the attachment process of the needle assembly 15 to the injection device 1, there will be sufficient time for the pressure relief action as described above. Insofar and immediately after having completed the attachment of the injection needle 19 to the injection device 1 the pressure relief has effectively terminated and the end user may even not become aware of the pressure relief action induced by the injection needle attachment.

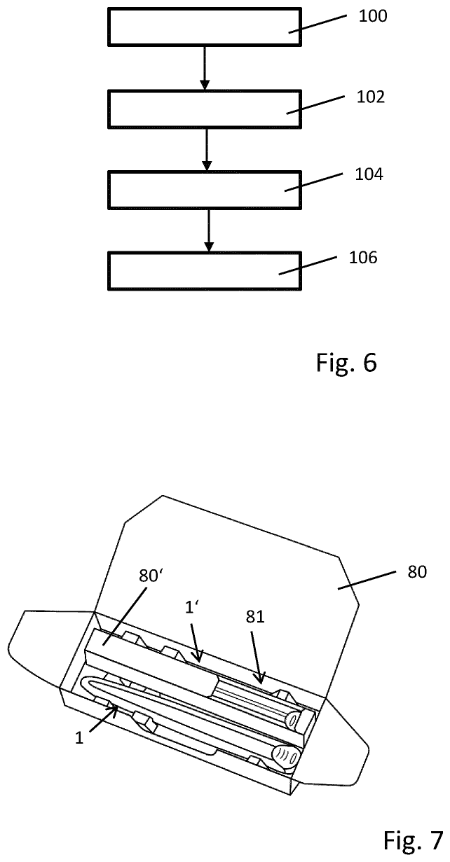

In the flowchart of FIG. 6 numerous steps of the method of preparing the injection device 1 are schematically illustrated. At a first step 100 there is provided an injection device 1 as described herein. The injection device 1 may be implemented as a pen-type injector of disposable or reusable type. The injection device 1 may be implemented all mechanically or even electromechanically. With some examples the injection device 1 includes a mechanical energy storage, such as a spring, which is biased initially and/or which is biased by and/or during setting of a dose.

With other examples the dispensing force for advancing the piston rod 20 in distal direction 2 for the purpose of injecting of a dose is entirely provided by a user of the device 1. With other examples the mechanical energy storage provides at least a part of the driving force during an injection procedure.

In a subsequent step 102 the injection device 1 is transferred into the pre-use configuration. In step 102 the piston rod 20 of the drive mechanism 8 is advanced in distal direction 2 so as to obtain a longitudinal and mechanically biasing abutment with the stopper 7 of the medicament container 6. With some exampled the piston rod 20 is advanced per default by a pre-defined distance relative to the housing 10 and/or relative to the medicament container 6.

With other examples the advancing motion may be pressure controlled. Here, a pressure sensing arrangement may be provided by way of which the abutment pressure between the stopper 7 and the piston rod 20 can be quantitatively determined. When reaching a predefined pre-use pressure between the piston rod 20 and the stopper 7 the pre-use configuration has been reached and an eventual ongoing distally advancing motion of the piston rod 20 is then terminated in response.

By moving the piston in distal direction 2 by a predefined distance the predefined distance is selected such that eventual tolerances, mechanical play and eventual longitudinal gap sizes between the piston rod and the stopper can be closed and/or eliminated. With some examples transferring of the injection device into the pre-use configuration is simply obtained by setting or dialing a predefined size of a dose, e.g. a dose size of only a few units and by conducting a respective dispensing procedure, e.g. by pressing the trigger 11 of the drive mechanism 8 thereby advancing the piston rod 20 or plunger by a well-defined longitudinal distance relative to the housing 10 and/or relative to the medicament container 6.

The advancing motion of the piston rod 20 is accompanied by the built-up of the pre-use pressure onto the stopper 17. When reaching the pre-use configuration the distal end 63 of the medicament container 6 is and remains sealed so as to prevent any uncontrolled expelling of the medicament therefrom. After step 102 and after having transferred the injection device 1 into the pre-use configuration it may be wrapped or packed in a packaging of may be subject to transportation and/or storage.

In a subsequent step 104, typically conducted by a consumer or end user of the injection device 1, the needle assembly 15 is duly attached to the injection device 1. Attaching of the needle assembly 15 includes penetration of the seal 26 by the injection needle 19 thereby providing a pressure relief of the drive mechanism 8 and/or of the medicament container 6. In the course of attaching the needle assembly 15 to the injection device 1, a respective amount of medicament 27 may be expelled through the injection needle 19. There may arise or evolve a few drops at the distal end 68 of the injection needle, which amount of medicament 27 may be trapped inside the inner needle cap 16.

In a subsequent and final step 106 of preparing the injection device 1 the end user may detach the outer needle cap 17 and the inner needle cap 16 so as to enable injection of the medicament into biological tissue.

in FIG. 7 there is further illustrated an injection system including at least one injection device 1, 1′ and a packaging 80, 80′. The packaging is implemented as a compound packaging and includes a receptacle 80 one sized to receive or to accommodate at least one injection device 1, 1′. In the presently illustrated example the packaging 80 is a secondary packaging or an outer packaging sized to receive two individual injection devices 1, 1′. The injection device 1′ is even wrapped in a further separate packaging 80′. The injection devices 1, 1′are both implemented as a pen-type injectors. They are arranged in the packagings 80, 80′ in the above-described pre-use configuration. For using the injection devices 1, 1′ it is only required to attach and/or to fix a needle assembly 15 to the outlet end of the respective injection devices 1, 1′.

Due to the mechanically biasing abutment between a stopper 7 of a medicament container 6 located inside the injection devices 1, 1′ and the respective piston rod 20 or pressure foot 23 there will be somewhat automatically conducted a procedure due to the pre-tensed or biased configuration of the drive mechanism 8 or drivetrain of the injection devices 1, 1′.

### REFERENCE NUMBERS

1 injection device
2 distal direction
3 proximal direction
4 dose incrementing direction
5 dose decrementing direction
6 medicament container
7 stopper
8 drive mechanism
10 housing
11 trigger
12 dose dial
13 dosage window
14 cartridge holder
15 needle assembly
16 inner needle cap
17 outer needle cap
18 protective cap
19 injection needle
20 piston rod
21 bearing
22 first thread
23 pressure foot
24 second thread
25 barrel
26 seal
27 medicament
28 threaded socket
30 drive sleeve
31 threaded section
32 flange
33 flange
35 last dose limiter
40 spring
44 clutch
50 number sleeve
54 stop face
55 aperture
60 fixing cap
61 outlet
62 outlet aperture
63 distal end
64 bulged portion
65 piercable portion
66 shoulder portion
67 proximal end
68 distal end
69 head portion
70 needle hub
80 packaging
81 receptacle

1-18. (canceled)

19. An injection device for setting and injecting a dose of a medicament, the injection device comprising:
a housing;
a medicament container containing the medicament and being sealed by a movable stopper in a proximal direction, the medicament container comprising an outlet at a distal end; and
a drive mechanism arranged inside the housing and comprising a piston rod extending along a longitudinal direction and being operable to exert a distally directed pressure onto the stopper for expelling the dose of the medicament through the outlet,
wherein in a pre-use configuration of the injection device, prior to expelling of a first dose of the medicament and/or prior to a first use of the injection device, the piston rod longitudinally abuts the stopper in a mechanically biased configuration.

20. The injection device of claim 19, wherein in the pre-use configuration, the piston rod exerts a distally directed pre-use pressure onto the stopper, and wherein the pre-use pressure is larger than or equal to a driving pressure required to move the stopper in a distal direction for injecting the dose of the medicament.

21. The injection device of claim 19, wherein in the pre-use configuration of the injection device, the outlet is sealed and is impenetrable by the medicament.

22. The injection device of claim 19, wherein a seal is fixed to the distal end by a fixing cap comprising an outlet aperture that provides access to a pierceable portion of the seal.

23. The injection device of claim 22, wherein in the pre-use configuration of the injection device, the pierceable portion of the seal comprises a bulged seal portion that at least partially protrudes distally through the outlet aperture.

24. The injection device of claim 19, wherein in the pre-use configuration of the injection device, a hydrostatic pressure inside the medicament container is larger than an atmospheric pressure.

25. The injection device of claim 19, wherein in the pre-use configuration of the injection device, the piston rod is in a priming position relative to the housing, and wherein the priming position is distally offset compared to an initial position of the piston rod upon assembly of the injection device.

26. The injection device of claim 19, wherein the injection device is transferable from the pre-use configuration into a ready-to-use configuration by attaching a needle assembly to the outlet, and wherein the needle assembly comprises an injection needle for penetrating a seal of the medicament container.

27. The injection device of claim 26, wherein the injection needle is a double-tipped injection needle that pierces the seal of the outlet of the medicament container when the injection device is transferred from the pre-use configuration into the ready-to-use configuration.

28. The injection device of claim 26, wherein the needle assembly comprises at least one of an outer needle cap and an inner needle cap that covers the distal end of the injection needle and that is configured to receive or to trap a portion of the medicament expelled through the distal end of the injection needle while the needle assembly is being attached to the injection device.

29. An injection system comprising:
an injection device for setting and injecting a dose of a medicament, the injection device comprising:
a housing,
a medicament container containing the medicament and being sealed by a movable stopper in a proximal direction, the medicament container comprising an outlet at a distal end, and
a drive mechanism arranged inside the housing and comprising a piston rod extending along a longitudinal direction and being operable to exert a distally directed pressure onto the stopper for expelling the dose of the medicament through the outlet,
wherein in a pre-use configuration of the injection device, prior to expelling of a first dose of the medicament and/or prior to a first use of the injection device, the piston rod longitudinally abuts the stopper in a mechanically biased configuration; and
a packaging providing a receptacle, wherein the injection device is arranged inside the receptacle in the pre-use configuration.

30. The injection system of claim 29, wherein in the pre-use configuration, the piston rod exerts a distally directed pre-use pressure onto the stopper, and wherein the pre-use pressure is larger than or equal to a driving pressure required to move the stopper in a distal direction for injecting of the dose of the medicament.

31. The injection system of claim 30, wherein in the pre-use configuration of the injection device, the outlet is sealed and is impenetrable by the medicament.

32. A method of preparing an injection device for injecting a medicament, the method comprising:
providing the injection device, the injection device comprising:
a housing,
a medicament container containing the medicament and being sealed by a movable stopper in a proximal direction, the medicament container comprising an outlet at a distal end, and
a drive mechanism arranged inside the housing and comprising a piston rod extending along a longitudinal direction and being operable to exert a distally directed pressure onto the stopper for expelling the dose of the medicament through the outlet,
wherein in a pre-use configuration of the injection device, prior to expelling of a first dose of the medicament and/or prior to a first use of the injection device, the piston rod longitudinally abuts the stopper in a mechanically biased configuration; and
transferring the injection device into the pre-use configuration by moving the piston rod in a distal direction against the stopper of the medicament container.

33. The method of claim 32, further comprising attaching a needle assembly to the injection device after transferring the injection device into the pre-use configuration, wherein the needle assembly comprises a double-tipped injection needle with a proximal end and a distal end.

34. The method of claim 33, wherein the proximal end penetrates the seal of the medicament container upon attaching the needle assembly to the injection device.

35. The method of claim 32, wherein while the needle assembly is being attached to the injection device, a portion of the medicament is expelled through the injection needle due to a pre-use pressure that is distally exerted onto the stopper by the piston rod in the pre-use configuration.

36. The method of claim 35, wherein the needle assembly comprises at least one of an outer needle cap and an inner needle cap that covers a distal end of the injection needle and receives or traps the portion of the medicament that is expelled through the distal end of the injection needle while the needle assembly is being attached to the injection device.

37. The method of claim 32, wherein the injection device is transferrable from the pre-use configuration into a ready-to-use configuration by attaching the needle assembly to the injection device.

38. The method of claim 32, wherein the injection device is arranged inside a packaging for transportation and/or storage after being transferred into the pre-use configuration.
